(19) United States
(12) Patent Application Publication
(10) Pub. No.: US 2013/0210655 A1
(43) Pub. Date: Aug. 15, 2013

(54) MICROARRAY WITH ENHANCED SPECIFICITY

(71) Applicant: SAMSUNG TECHWIN CO., LTD., (US)

(72) Inventors: Song Chun CHONG, Ellicott City, MD (US); John HARVEY, Elkridge, MD (US)

(73) Assignee: SAMSUNG TECHWIN CO., LTD., Changwon-city (KR)

(21) Appl. No.: 13/721,913

(22) Filed: Dec. 20, 2012

Related U.S. Application Data
(60) Provisional application No. 61/579,133, filed on Dec. 22, 2011.

(51) Int. Cl. C12Q 1/68

(52) U.S. Cl. CPC C12Q 1/683
USPC 506/9; 435/6.11; 506/16

(57) ABSTRACT

Provided is an array including a plurality of target specific array nucleic acid molecules wherein each target specific array nucleic acid molecule contains: (i) a 5′-end DNA segment, (ii) a 3′-end DNA segment, and (iii) an RNA sequence of 1-6 bases within the molecule; and wherein each of the 5-end of the target specific array nucleic acid molecules is coupled to a detectable label. A method for detecting a target nucleic acid and a method for detecting polymorphism in a target nucleic using the array are also provided.

### BACKGROUND

Rapid and reliable detection of genetic mutations in a patient's DNA can guide a physician's clinical diagnosis and choice of medical treatment. This is particularly true in oncology where the presence of discrete mutations within the coding regions of oncogenes can be powerful predictors of cancer susceptibility and prognosis.

For example, genetic testing for the presence of harmful mutations within the BRCA tumor suppressor genes are is now routine in most medical practices. Harmful BRCA-1 mutations can greatly increase a woman's risk of developing breast and/or ovarian cancer at an early age (before menopause) as well as the risk of developing cervical, uterine, pancreatic, and colon cancer. Harmful BRCA2 mutations may additionally increase the risk of pancreatic cancer, stomach cancer, gallbladder and bile duct cancer, and melanoma. Mutations in several other genes, including TP53, PTEN, STK11/LKB1, CDH1, CHEK2, ATM, MLH1, and MSH2, have been associated with hereditary breast and/or ovarian tumors.

With the advent of nucleic acid amplification, as little as a single molecule of any DNA sequence can be copied a sufficient number of times to permit SNP sequence analysis. SNPs may be detected by a variety of techniques, such as DNA sequencing, fluorescent probe detection, mass spectrometry or DNA microarray hybridization (e.g., U.S. Pat. Nos. 5,885,775; 6,368,799). Many of these procedures remain inadequate however for high throughput applications because of either overall poor sensitivity, cost, time expenditure or the need for post-PCR processing. Existing methods of SNP detection also have an unacceptably high level of false positive and/or false negative results.

Traditional microarray analysis of gene expression relies on the hybridization between labeled probe DNA or RNA sequences and unlabeled target sequences that are sequestered on a physical matrix such as glass. These types of arrays can be used to quantify the level of expression of multiple genes from the same source under different physiological conditions or as a means to identify a specific nucleic acid target within an ensemble of related or unrelated sequences. There are numerous examples of the latter application including both genetic polymorphism analysis and identification of bacterial, viral, or fungal contamination in a test sample.

In general, nucleic acid hybridization involves contact between single strands of probe and target sequences. After allowing single strands of probe with single strands of target are brought to be in contact with each other, strands with complementary base sequences can be allowed to reassociate (also called hybridization or annealing). The annealing of a labeled probe strand and a complementary unlabeled target strand leads to the formation of a labeled probe-target heteroduplex. The rationale of the hybridization assay is to use the labeled probe to query the target sequence by identifying fragments in the complex target which may be related in sequence to the probe.

A useful measure of the stability of a probe-target heteroduplex is the melting temperature (Tm). This is the temperature corresponding to the midpoint in the observed transition from double-stranded to single-stranded form. Conveniently, this transition can be detected by measuring the optical density of the heteroduplex. The bases of the nucleic acids absorb 260 nm ultraviolet (UV) light strongly. However, the absorption by double-stranded DNA is considerably less than that of the free nucleotides. This difference, the so-called hypochromic effect, is due to interactions between the electron systems of adjacent bases, arising from the way in which adjacent bases are stacked in parallel in a double helix. If duplex DNA is gradually heated, therefore, there will be an increase in the optical density (OD) at 260 nm towards the value characteristic of the free bases. The temperature at which there is a midpoint in the optical density shift is then taken as the Tm.

Probe-target heteroduplexes are most stable thermodynamically when the region of duplex formation contains perfect base matching. Mismatches between the two strands of a heteroduplex reduce the Tm. In general, each 1% of mismatching in, for example a DNA duplex, reduces the Tm by approximately 1° C.

An effective microarray analysis often requires an optimization of appropriate conditions specific to their platform such as hybridization time, temperature, and washing stringency. This is especially true in the case of single nucleotide polymorphism (SNP) analysis where, with the exception of a single nucleotide, the probe and target are fully complementary. Optimizing hybridization conditions can be difficult because determining the results of changing one condition requires a complete repetition of the microarray experiment. The amount and distribution of the target nucleic acid present on the array is fixed by the array platform. The user controls the amount of labeled sample probe incubated with the array. The amount of labeled probe used in a microarray experiment depends on balancing two opposing considerations.

1. On one hand, the hybridization of more labeled probe increases signal intensity and decreases the number of false negatives.

2. On the other hand, increasing amount of the labeled probe may cause signal saturation and lead to an increase in the number of false positives.

### SUMMARY

a) In an embodiment, a method and a kit for detecting a target nucleic acid molecule in a sample is disclosed, the method including: (a) providing an array comprising a plurality of probe specific targets wherein each probe specific target comprises: (i) a 5′-end DNA segment, (ii) a 3′-end DNA segment, and (iii) an RNA sequence within the molecule; and wherein each of the 5-ends of the probe specific target array nucleic acid molecules is coupled to a detectable label, said DNA and RNA segments of the target being substantially complimentary to the probe nucleic acid, wherein the RNA sequence of the probe specific target is capable of being cleaved by the cleaving agent and a cleavage of the RNA sequence in the probe specific target results in an emission of a detectable signal from the label; (b) bringing a sample containing probe nucleic to be contacted with the probe specific target array probes in the presence of a mixture containing a reaction buffer and cleaving agent under conditions where the RNA sequence within the probe specific target forms a RNA:DNA heteroduplex with the complimentary sequence in the probe nucleic acid; and (c) detecting a change in the emission of a signal from the label of the probe specific target are provided.

In another embodiment, a method of detecting a target nucleic acid molecule in a sample is disclosed, the method includes the steps of: (a) providing an array comprising a plurality of probe specific targets wherein each probe specific target comprises: (i) a 5′-end DNA segment, (ii) a 3′-end DNA segment, and (iii) an RNA sequence within the molecule; and wherein each of the 5-end of the probe specific target array nucleic acid molecules is coupled to a detectable label, said DNA and RNA segments of the probe specific target being substantially complimentary to the probe nucleic acid, wherein the RNA sequence of the probe specific target is capable of being cleaved by the cleaving agent and a cleavage of the RNA sequence in the probe specific target results in an emission of a detectable signal from the label; (b) bringing a sample to be contact with the array of probe specific targets in the presence of a mixture containing a first primer oligonucleotide, a second primer oligonucleotide, a polymerase activity, a cleaving agent, and deoxynucleoside triphosphates wherein the first primer oligonucleotide and the second oligonucleotide can anneal to the probe nucleic acid, under conditions where the RNA sequence within the probe specific target forms a RNA:DNA heteroduplex with the complimentary sequence in the probe nucleic acid; and (c) detecting a change in the emission of a signal from the label of the probe specific target.

In one embodiment, there is disclosed a method for the real-time detection of a polymorphism in a probe DNA, comprising the steps of providing a sample to be tested for the presence of a probe DNA, providing a pair of amplification primers that can anneal to the probe DNA, wherein a first amplification primer anneals upstream of the location of the polymorphism and the second amplification primer anneals downstream of the location of the polymorphism, providing a probe specific target comprising a detectable label and DNA and RNA nucleic acid sequences, wherein the probe specific targets RNA nucleic acid sequences are entirely complementary to a selected region of the probe DNA and the probe specific targets DNA nucleic acid sequences are substantially complementary to DNA sequences adjacent to the selected region of the probe DNA sequence, amplifying a PCR fragment between the first and second amplification primers in the presence of an amplifying polymerase activity, amplification buffer; an RNase H activity and the probe under conditions where the RNA sequences within the probe specific target can form a RNA:DNA heteroduplex with the complementary DNA sequences in the probe PCR fragment, and detecting a real-time increase in the emission of a signal from the label on the probe specific target, wherein the increase in signal indicates the presence of the probe DNA, wherein the probe specific target is immobilized on a surface of a solid support.

In one aspect, the real-time increase in the emission of the signal from the label on the probe specific target results from the RNase H cleavage of the probe specific targets RNA sequences in the RNA:DNA heteroduplex.

In another embodiment, there is disclosed a method for the real-time detection of a polymorphism in a probe DNA, comprising steps of providing a sample to be tested for the presence of a probe DNA having a polymorphism, providing a pair of amplification primers that can anneal to the probe DNA, wherein a first amplification primer anneals upstream of the location of the polymorphism and the second amplification primer anneals downstream of the location of the polymorphism, providing a probe specific target comprising a detectable label and DNA and RNA nucleic acid sequences, wherein the probe specific targets RNA nucleic acid sequences are entirely complementary to a selected region of the probe DNA sequence comprising a wild type DNA sequence at the location of the polymorphism and the probe specific targets DNA nucleic acid sequences are substantially complementary to DNA sequences adjacent to the selected region of the probe DNA sequence, amplifying a PCR fragment between the first and second amplification primers in the presence of an amplifying polymerase activity, amplification buffer; an RNase H activity and the probe under conditions where the RNA sequences within the probe specific target can form a RNA:DNA heteroduplex with the complementary DNA sequences in the probe PCR fragment comprising the polymorphism, and detecting a real-time decrease in the emission of a signal from the label on the probe specific target, wherein the decrease in signal indicates the presence of the polymorphism in the probe DNA, wherein the probe specific target is immobilized on a surface of a solid support.

In another embodiment, there is disclosed a kit for the real-time detection of a polymorphisms in a probe DNA comprising a pair of amplification primers that can anneal to a probe DNA, wherein a first amplification primer anneals upstream of the location of a polymorphism and a second amplification primer anneals downstream of the location of the polymorphism, a plurality of probe specific targets each comprising a detectable label and DNA and RNA nucleic acid sequences, wherein the probe specific targets RNA nucleic acid sequences are entirely complementary to a selected region of the probe DNA sequence comprising the polymorphism and the probe specific targets DNA nucleic acid sequences are substantially complementary to probe DNA sequences adjacent to the selected region of the probe DNA sequence; and an RNase H activity, wherein the probe specific targets are immobilized on a surface of a solid support. The kit may further comprise an amplifying polymerase activity, and an amplification buffer.

In another embodiment, there is disclosed a kit for the real-time detection of a polymorphism in a probe DNA comprising a pair of amplification primers that can anneal to a probe DNA, wherein a first amplification primer anneals upstream of the location of a polymorphism and a second amplification primer anneals downstream of the location of the polymorphism, a plurality of probe specific targets each comprising a detectable label and DNA and RNA nucleic acid sequences, wherein the probe specific target RNA nucleic acid sequences are entirely complementary to a selected region of the probe DNA sequence comprising the wild type DNA sequence at the location of the polymorphism and the probe specific targets DNA nucleic acid sequences are substantially complementary to DNA sequences adjacent to the selected region of the probe DNA sequence; and an RNase H activity, wherein the probe specific targets are immobilized on a surface of a solid support. The kit may further comprise an amplifying polymerase activity, and an amplification buffer.

The detectable label on the probe specific target can be a fluorescent label such as a FRET pair.

The amplifying polymerase activity may be an activity of a thermostable DNA polymerase and the site-specific RNase H activity may be the activity of a thermostable RNase H or a hot start thermostable RNase H activity.

In another embodiment, an array is disclosed, wherein the array includes a plurality of probe specific target nucleic acid molecules wherein each probe specific target nucleic acid molecule comprises: (i) a 5′-end DNA segment, (ii) a 3′-end DNA segment, and (iii) an RNA sequence of 1-6 bases within the molecule; and wherein each of the 5-end of the probe specific target nucleic acid molecules is labeled with a detectable marker

### BRIEF DESCRIPTION OF DRAWINGS

FIG. 1 is a schematic illustration of an array containing two different probe specific target sequences (target Nos. 1-10 and target Nos. 11-20), each in various concentrations.

FIG. 2A and 2B are graphs showing the results explained in the Experiment section. In FIGS. 2A and 2B baseline fluorescence from the negative control (Initial signal) was subtracted from the probe containing sample (Final) to calculate the corrected signal (Delta).

### DETAILED DESCRIPTION OF EMBODIMENTS

The practice of the embodiments described herein employs, unless otherwise indicated, conventional molecular biological techniques within the skill of the art. Such techniques are well known to the skilled worker, and are explained fully in the literature. See, e.g., Ausubel, et al., ed., Current Protocols in Molecular Biology, John Wiley & Sons, Inc., NY, N.Y. (1987-2008), including all supplements; Sambrook, et al., Molecular Cloning: A Laboratory Manual, 2nd Edition, Cold Spring Harbor, N.Y. (1989).

Unless defined otherwise, all technical and scientific terms used herein have the same meaning as is commonly understood by one of skill in the art. The specification also provides definitions of terms to help interpret the disclosure and claims of this application. In the event a definition is not consistent with definitions elsewhere, the definition set forth in this application will control.

As used herein, the term “nucleic acid” refers to an oligonucleotide or polynucleotide, wherein said oligonucleotide or polynucleotide may be modified or may comprise modified bases. Oligonucleotides are single-stranded polymers of nucleotides comprising from 2 to 60 nucleotides. Polynucleotides are polymers of nucleotides comprising two or more nucleotides. Polynucleotides may be either double-stranded DNAs, including annealed oligonucleotides wherein the second strand is an oligonucleotide with the reverse complement sequence of the first oligonucleotide, single-stranded nucleic acid polymers comprising deoxythymidine, single-stranded RNAs, double stranded RNAs or RNA/DNA heteroduplexes. Nucleic acids include, but are not limited to, genomic DNA, cDNA, hnRNA, snRNA, mRNA, rRNA, tRNA, fragmented nucleic acid, nucleic acid obtained from subcellular organelles such as mitochondria or chloroplasts, and nucleic acid obtained from microorganisms or DNA or RNA viruses that may be present on or in a biological sample. Nucleic acids may be composed of a single type of sugar moiety, e.g., as in the case of RNA and DNA, or mixtures of different sugar moieties, e.g., as in the case of RNA/DNA chimeras.

A “probe DNA or “probe RNA” or “ probe nucleic acid,” or “probe nucleic acid sequence” refers to a nucleic acid that is targeted by DNA amplification. A probe nucleic acid sequence serves as a template for amplification in a PCR reaction or reverse transcriptase-PCR reaction. Probe nucleic acid sequences may include both naturally occurring and synthetic molecules. Exemplary probe nucleic acid sequences include, but are not limited to, genomic DNA or genomic RNA.

As used herein, “label” or “detectable label” can refer to any chemical moiety attached to a nucleotide, nucleotide polymer, or nucleic acid binding factor, wherein the attachment may be covalent or non-covalent. Preferably, the label is detectable and renders said nucleotide or nucleotide polymer detectable to the practitioner of the invention. Detectable labels include luminescent molecules, chemiluminescent molecules, fluorochromes, fluorescent quenching agents, colored molecules, radioisotopes or scintillants. Detectable labels also include any useful linker molecule (such as biotin, avidin, streptavidin, HRP, protein A, protein G, antibodies or fragments thereof, Grb2, polyhistidine, Ni.sup.2+, FLAG tags, myc tags), heavy metals, enzymes (examples include alkaline phosphatase, peroxidase and luciferase), electron donors/acceptors, acridinium esters, dyes and calorimetric substrates. It is also envisioned that a change in mass may be considered a detectable label, as is the case of surface plasmon resonance detection. The skilled artisan would readily recognize useful detectable labels that are not mentioned above, which may be employed in the operation of the present invention.

As used herein, the term “oligonucleotide” is used sometimes interchangeably with “primer” or “polynucleotide.” The term “primer” refers to an oligonucleotide that acts as a point of initiation of DNA synthesis in a PCR reaction. A primer is usually about 15 to about 35 nucleotides in length and hybridizes to a region complementary to the target sequence.

Oligonucleotides may be synthesized and prepared by any suitable methods (such as chemical synthesis), which are known in the art. Oligonucleotides may also be conveniently available through commercial sources.

“Polymerase chain reaction,” or “PCR,” generally refers to a method for amplification of a desired nucleotide sequence in vitro. The procedure is described in detail in U.S. Pat. Nos. 4,683,202, 4,683,195, 4,800,159, and 4,965,188, the contents of which are hereby incorporated herein in their entirety. Generally, the PCR process consists of introducing a molar excess of two or more extendable oligonucleotide primers to a reaction mixture comprising the desired target sequence(s), where the primers are complementary to opposite strands of the double stranded target sequence. The reaction mixture is subjected to a program of thermal cycling in the presence of a DNA polymerase, resulting in the amplification of the desired target sequence flanked by the DNA primers.

The terms “annealing” and “hybridization” are used interchangeably and mean the base-pairing interaction of one nucleic acid with another nucleic acid that results in formation of a duplex, triplex, or other higher-ordered structure. In certain embodiments, the primary interaction is base specific, e.g., A/T and G/C, by Watson/Crick and Hoogsteen-type hydrogen bonding. In certain embodiments, base-stacking and hydrophobic interactions may also contribute to duplex stability.

As used herein, the term “substantially complementary” refers to two nucleic acid strands that are sufficiently complimentary in sequence to anneal and form a stable duplex. The complementarity does not need to be perfect; there may be any number of base pair mismatches, for example, between the two nucleic acids. However, if the number of mismatches is so great that no hybridization can occur under even the least stringent hybridization conditions, the sequence is not a substantially complementary sequence. When two sequences are referred to as “substantially complementary” herein, it means that the sequences are sufficiently complementary to each other to hybridize under the selected reaction conditions. The relationship of nucleic acid complementarity and stringency of hybridization sufficient to achieve specificity is well known in the art. Two substantially complementary strands can be, for example, perfectly complementary or can contain from 1 to many mismatches so long as the hybridization conditions are sufficient to allow, for example discrimination between a pairing sequence and a non-pairing sequence. Accordingly, “substantially complementary” sequences can refer to sequences with base-pair complementarity of 100, 95, 90, 80, 75, 70, 60, 50 percent or less, or any number in between, in a double-stranded region.

Determining the appropriate hybridization conditions for each experiment is time consuming and may be expensive for specialty microarrays that are custom printed and contain large numbers of targets. It is therefore desirable to develop new methods that reduce the need to optimize the hybridization time, temperature, and probe-target ratio. Ideally, this technology would also greatly reduce the need for washing stringency.

The term “polymorphism” refers to the occurrence of two or more alternative genomic sequences or alleles between or among different genomes or individuals.

The term “polymorphic” refers to the condition in which two or more variants of a specific genomic sequence are found in a population.

The term “polymorphic site” is the locus at which the variation occurs. A polymorphic site generally has at least two alleles, each occurring at a significant frequency in a selected population. A polymorphic locus may be as small as one base pair, in which case it is referred to as single nucleotide polymorphism (SNP). The first identified allelic form is arbitrarily designated as the reference, wild-type, common or major form, and other allelic forms are designated as alternative, minor, rare or variant alleles.

The term “genotype” refers to a description of the alleles of a gene contained in an individual or sample.

The term “single nucleotide polymorphism” (“SNP”) refers to a site of one nucleotide that varies between alleles. Single nucleotides may be changed (substitution), removed (deletions) or added (insertion) to a polynucleotide sequence. Insertion or deletion SNPs may cause a translational frameshift. Single nucleotide polymorphisms may fall within coding sequences of genes, non-coding regions of genes, or in the intergenic regions between genes. SNPs within a coding sequence will not necessarily change the amino acid sequence of the protein that is produced, due to degeneracy of the genetic code. A SNP in which both forms lead to the same polypeptide sequence is termed synonymous (sometimes called a silent mutation) but if a different polypeptide sequence is produced they are nonsynonymous. A nonsynonymous change may either be mis sense or nonsense, where a missense change results in a different amino acid, while a nonsense change results in a premature stop codon. “Functional SNPs” are SNPs that produce alterations in gene expression or in the expression or function of a gene product, and therefore are most predictive of a possible clinical phenotype. The alterations in gene function caused by functional SNPs may include changes in the encoded polypeptide, changes in mRNA stability, binding of transcriptional and translation factors to the DNA or RNA, and the like. SNPs that are not in protein-coding regions may still have consequences for gene splicing, transcription factor binding, or the sequence of non-coding RNA.

In accordance with an embodiment, one of a skilled artisan understands that SNPs have two alternative alleles, each corresponds to a nucleotide that may exist in the chromosome. Thus, a SNP is characterized by two nucleotides out of four (A, C, G, T). An example would be that a SNP has either allele C or allele T at a given position on each chromosome. This is shown as C>T or C/T. The more commonly occurring allele is shown first (in this case, it is C) and called the major, common or wild-type allele. The alternative allele that occurs less commonly instead of the common allele (in this case, it is T) is called minor, rare or variant allele. Wild-type and variant alleles may be referred to as common and rare alleles respectively. Since humans are diploid organisms meaning that each chromosome occurs in two copies, each individual has two alleles at a SNP. These alleles may be two copies of the same allele (CC or TT) or they may be different ones (CT). The CC, CT and TT are called genotypes. Among these CC and TT are characterized by having two copies of the same allele and are called homozygous genotypes. The genotype CT has different alleles on each chromosome and is a heterozygous genotype. Individuals bearing homozygote or heterozygote genotypes are called homozygous and heterozygous, respectively.

### Selection of SNPs

The term “polymorphism” refers to the occurrence of two or more alternative genomic sequences or alleles between or among different genomes or individuals.

The term “polymorphic” refers to the condition in which two or more variants of a specific genomic sequence are found in a population.

The term “polymorphic site” is the locus at which the variation occurs. A polymorphic site generally has at least two alleles, each occurring at a significant frequency in a selected population. A polymorphic locus may be as small as one base pair, in which case it is referred to as single nucleotide polymorphism (SNP). The first identified allelic form is arbitrarily designated as the reference, wild-type, common or major form, and other allelic forms are designated as alternative, minor, rare or variant alleles.

The term “genotype” refers to a description of the alleles of a gene contained in an individual or sample.

The term “single nucleotide polymorphism” (“SNP”) refers to a site of one nucleotide that varies between alleles. Single nucleotides may be changed (substitution), removed (deletions) or added (insertion) to a polynucleotide sequence. Insertion or deletion SNPs may cause a translational frameshift. Single nucleotide polymorphisms may fall within coding sequences of genes, non-coding regions of genes, or in the intergenic regions between genes. SNPs within a coding sequence will not necessarily change the amino acid sequence of the protein that is produced, due to degeneracy of the genetic code. A SNP in which both forms lead to the same polypeptide sequence is termed synonymous (sometimes called a silent mutation) but if a different polypeptide sequence is produced they are nonsynonymous. A nonsynonymous change may either be mis sense or nonsense, where a missense change results in a different amino acid, while a nonsense change results in a premature stop codon. “Functional SNPs” are SNPs that produce alterations in gene expression or in the expression or function of a gene product, and therefore are most predictive of a possible clinical phenotype. The alterations in gene function caused by functional SNPs may include changes in the encoded polypeptide, changes in mRNA stability, binding of transcriptional and translation factors to the DNA or RNA, and the like. SNPs that are not in protein-coding regions may still have consequences for gene splicing, transcription factor binding, or the sequence of non-coding RNA.

In accordance with an embodiment, one of a skilled artisan understands that SNPs have two alternative alleles, each corresponds to a nucleotide that may exist in the chromosome. Thus, a SNP is characterized by two nucleotides out of four (A, C, G, T). An example would be that a SNP has either allele C or allele T at a given position on each chromosome. This is shown as C>T or C/T. The more commonly occurring allele is shown first (in this case, it is C) and called the major, common or wild-type allele. The alternative allele that occurs less commonly instead of the common allele (in this case, it is T) is called minor, rare or variant allele. Wild-type and variant alleles may be referred to as common and rare alleles respectively. Since humans are diploid organisms meaning that each chromosome occurs in two copies, each individual has two alleles at a SNP. These alleles may be two copies of the same allele (CC or TT) or they may be different ones (CT). The CC, CT and TT are called genotypes. Among these CC and TT are characterized by having two copies of the same allele and are called homozygous genotypes. The genotype CT has different alleles on each chromosome and is a heterozygous genotype. Individuals bearing homozygote or heterozygote genotypes are called homozygous and heterozygous, respectively. Further information of detecting SNP using a probe specific target having a DNA sequence-RNA sequence-DNA sequence may be found in copending application Ser. No. 13/158,593, of which content is incorporated by reference herein. According to an embodiment, a microarray method employing a probe specific target endonuclease is provided. The probe specific target endonuclease can distinguish completely paired probe-target heteroduplexes from partially paired probe-target heteroduplexes based on factors other than pure hydrogen bonding.

In this scenario the normal microarray actors are reversed so that the probe specific target is labeled and affixed to a solid substrate, while the probe is unlabeled. In addition, the probe specific target has a chimeric DNA-RNA-DNA structure that can act as a substrate for the endonuclease. Such DNA-RNA-DNA structure of a probe specific target (sometimes, referred to as “CataCleave probe”) technology is described in U.S. Pat. No. 5,763,181, of which entire disclosure is incorporated herein by reference.

In an embodiment, the probe specific target sequence may have a structure of DNA-RNA-DNA in its molecule. The probe specific target may be attached with a detectable label at their 5- and 3-ends or internally. The detectable label may be a pair of a pair of fluorescent donor and quencher, each attached to a DNA portion of the probe specific target with a sufficient distance from each other.

As used herein, “label” or “detectable label” can refer to any chemical moiety attached to a nucleotide, nucleotide polymer, or nucleic acid binding factor, wherein the attachment may be covalent or non-covalent. Preferably, the label is detectable and renders said nucleotide or nucleotide polymer detectable to the practitioner of the invention. Detectable labels include luminescent molecules, chemiluminescent molecules, fluorochromes, fluorescent quenching agents, colored molecules, radioisotopes or scintillants. Detectable labels also include any useful linker molecule (such as biotin, avidin, streptavidin, HRP, protein A, protein G, antibodies or fragments thereof, Grb2, polyhistidine, Ni²⁺, FLAG tags, myc tags), heavy metals, enzymes (examples include alkaline phosphatase, peroxidase and luciferase), electron donors/acceptors, acridinium esters, dyes and calorimetric substrates. It is also envisioned that a change in mass may be considered a detectable label, as is the case of surface plasmon resonance detection. The skilled artisan would readily recognize useful detectable labels that are not mentioned above, which may be employed in the operation of the present invention. In an embodiment, the label is a FRET (fluorescence resonance energy transfer) label.

In one example of a FRET assay, a molecular beacon is a single stranded oligonucleotide designed so that in the unbound state the probe forms a secondary structure where the donor and acceptor chromophores are in close proximity and donor emission is reduced. At the proper reaction temperature the beacon unfolds and specifically binds to the amplicon. Once unfolded the distance between the donor and acceptor chromophores increases such that FRET is reversed and donor emission can be monitored using specialized instrumentation. That is, donor emission is quenched in the absence of a complementary sequence by FRET between two chromophores. The donor chromophore, in its excited state, may transfer energy to an acceptor chromophore when the pair is in close proximity. This transfer is always non-radiative and occurs through dipole-dipole coupling. Any process that sufficiently increases the distance between the chromophores will decrease FRET efficiency such that the donor chromophore emission can be detected radiatively. Common donor chromophores include FAM, TAMRA, VIC, JOE, Cy3, Cy5, and Texas Red. Acceptor chromophores are chosen so that their excitation spectra overlap with the emission spectrum of the donor. An example of such a pair is FAM-TAMRA. There are also non fluorescent acceptors that will quench a wide range of donors. Other examples of appropriate donor-acceptor FRET pairs will be known to those skilled in the art.

### Array

The term “array” as used herein refers to an intentionally created collection of molecules which can be prepared either synthetically or biosynthetically. The molecules in the array can be identical or different from each other. The array can assume a variety of formats, for example, libraries of soluble molecules; libraries of compounds tethered to resin beads, silica chips, or other solid supports.

In an embodiment employing a solid support, the donor containing portion of the probe specific target may be attached to a predetermined region of a solid support either covalently or non-covalently.

A “predefined region” is a localized area on a substrate which is, was, or is intended to be used for formation of a selected polymer and is otherwise referred to herein in the alternative as “reaction” region, a “selected” region, simply a “region” or a “feature”. The predefined region may have any convenient shape, e.g., circular, rectangular, elliptical, wedge-shaped, etc. In accordance with the present invention, the arrays of the present invention have features on the order of 10-100 μm, i.e. 10×10 μm² to 100×100 μm² for approximately square features. More preferably the features will be on the order of 1-10 μm. In preferred aspects the present invention may be used in combination with arrays having features having sub-micron dimensions. Such features are preferably on the order of 100-1000 nm. Within these regions, the polymer synthesized therein is preferably synthesized in a substantially pure form. However, in other embodiments of the invention, predefined regions may substantially overlap. In such embodiments, hybridization results may be resolved by software, for example. Smaller feature sizes allow larger numbers of features on arrays of a given size. For example, a about 1.3 million features can be included using 11×11 μm features on a 1.28×1.28 cm array and the same array can have more then 6 million features with 5×5 μm features or more than 2 million with 8×8 μm features. Using a 5×5 inch wafer 49 such arrays can be synthesized on a wafer, for about 63.7 million features on a wafer. The wafer can be diced to form arrays of a variety of sizes, for example, 20×20 dicing of the wafer gives 400 arrays and 30×30 gives 900 arrays. One of skill in the art will recognize that larger wafers may also be used and smaller feature size allows larger numbers of features in a given area. Features sizes that may be used include 5×5 μm² or 25 μm² and 1×1 μm (1 μm²) and smaller, for example, 0.5×0.5 μm (0.25 μm²) features. The methods contemplate arrays of 1 to 2, 2-5, 5-10, 10-20, or 20-100 million different features, each feature containing many copies of a probe specific target sequence.

The term “solid support”, “support”, and “substrate” as used herein are used interchangeably and refer to a material or group of materials having a rigid or semi-rigid surface or surfaces. In many embodiments, at least one surface of the solid support will be substantially flat, although in some embodiments it may be desirable to physically separate synthesis regions for different compounds with, for example, wells, raised regions, pins, etched trenches, or the like. According to other embodiments, the solid support(s) will take the form of beads, resins, gels, microspheres, or other geometric configurations. See U.S. Pat. No. 5,744,305 for exemplary substrates.

DNA arrays can be manufactured with DNA molecules immobilized on the predefined region of the solid support.

The probe specific target molecules to be spotted are between 20-50 nucleotides in length and have a melting temperature of between 45° C. and 75° C. The molecules contain a sequence of 1-4 ribonucleotides that can be cleaved by a cleaving agent. Either the 5′ or 3′ end is attached to the substrate by a covalent or non-covalent linkage. Ideally, the 5′ or 3′ end of the molecule will contain a C4-C18 or longer spacer arm to reduce steric interference with the surface of the substrate and enhance probe binding. The end of the target molecule that is attached to the substrate is labeled with the donor moiety of the donor-quencher FRET pair. For other types of detectable markers the marker is attached to the end attached to the substrate. Multiplex detection can be provided either spatially, where each spot contains a ensemble of identical probe specific target molecules and identity is dependent on location, or by labeling the probe specific target molecules with multiple detectable markers and identity is dependent on the unique characteristics of each detectable marker.

### RNase

The reaction includes an endonuclease, for example RNase H, in particular RNase HI enzyme that will specifically cleave the RNA sequence portion of a fully complementary RNA-DNA duplex.

The probe is provided either from the sample without amplification or by reverse transcription to convert RNA to cDNA. In addition, the number of probes can also be enriched by a method such as PCR. When the probe and probe specific target are allowed to be in contact with each other under permissive conditions they will hybridize to form a heteroduplex. When the probe specific target is a deoxynucleic acid molecule, while most of the heteroduplex will be DNA-DNA a small region will also form a RNA-DNA heteroduplex that is to be subject to RNA endonuclease cleavage. The length of this region may be 1-6 bases. In other embodiment, the length may be 1-4 bases. After cleavage, the quencher containing portion of the probe specific target and probe dissociate from the complex of the heteroduplex and are released into the reaction buffer, which will increase the donor emission within target area. Since the probe is not cleaved by RNase HI it may be recycled and bind to other targets. In this way it is possible for a single probe to serve as a locus to initiate multiple rounds of target cleavage, resulting in signal amplification. Once complete the microarray is read and the total intensity within each probe specific target area is calculated. Emission intensity will increase over background in proportion to the number of probes present in the sample. RNase H hydrolyzes RNA in RNA-DNA hybrids. This enzyme was first identified in calf thymus but has subsequently been described in a variety of organisms. RNase H activity appears to be ubiquitous in eukaryotes and bacteria. Although RNase H's constitute a family of proteins of varying molecular weight and nucleolytic activity, substrate requirements appear to be similar for the various isotypes. For example, most RNase H's studied to date function as endonucleases and requiring divalent cations (e.g., Mg²⁺, Mn²⁺) to produce cleavage products with 5′ phosphate and 3′ hydroxyl termini.

RNase HI from E. coli is the best-characterized member of the RNase H family. In addition to RNase HI, a second E. coli RNase H, RNase HII has been cloned and characterized (Itaya, M., Proc. Natl. Acad. Sci. USA, 1990, 87, 8587-8591). It is comprised of 213 amino acids while RNase HI is 155 amino acids long. E. coli RNase HIM displays only 17% homology with E. coli RNase HI. An RNase H cloned from S. typhimurium differed from E. coli RNase HI in only 11 positions and was 155 amino acids in length (Itaya, M. and Kondo K., Nucleic Acids Res., 1991, 19, 4443-4449).

Proteins that display RNase H activity have also been cloned and purified from a number of viruses, other bacteria and yeast (Wintersberger, U. Pharmac. Ther., 1990, 48, 259-280). In many cases, proteins with RNase H activity appear to be fusion proteins in which RNase H is fused to the amino or carboxy end of another enzyme, often a DNA or RNA polymerase. The RNase H domain has been consistently found to be highly homologous to E. coli RNase HI, but because the other domains vary substantially, the molecular weights and other characteristics of the fusion proteins vary widely.

In higher eukaryotes two classes of RNase H have been defined based on differences in molecular weight, effects of divalent cations, sensitivity to sulfhydryl agents and immunological cross-reactivity (Busen et al., Eur. J. Biochem., 1977, 74, 203-208). RNase HI enzymes are reported to have molecular weights in the 68-90 kDa range, be activated by either Mn²⁺ or Mg²⁺ and be insensitive to sulfhydryl agents. The embodiments disclosed herein have the following advantages, among others:

1. Increase in the usable Tm range for probe-target hybridization because RNase HI is used to discriminate base mismatches in addition to hydrogen bonding. This allows for greater flexibility in designing the probe specific target.

2. Increased sensitivity because each probe serves as a locus for many rounds of probe specific target cleavage. This helps to reduce the number of false negatives due to low probe availability.

3. Large reduction in false positives due to target saturation with probe. With the CataCleave reaction heteroduplex formation does not lead directly to signal production. Target cleavage is required for signal production so that mass action hybridization effects are minimized.

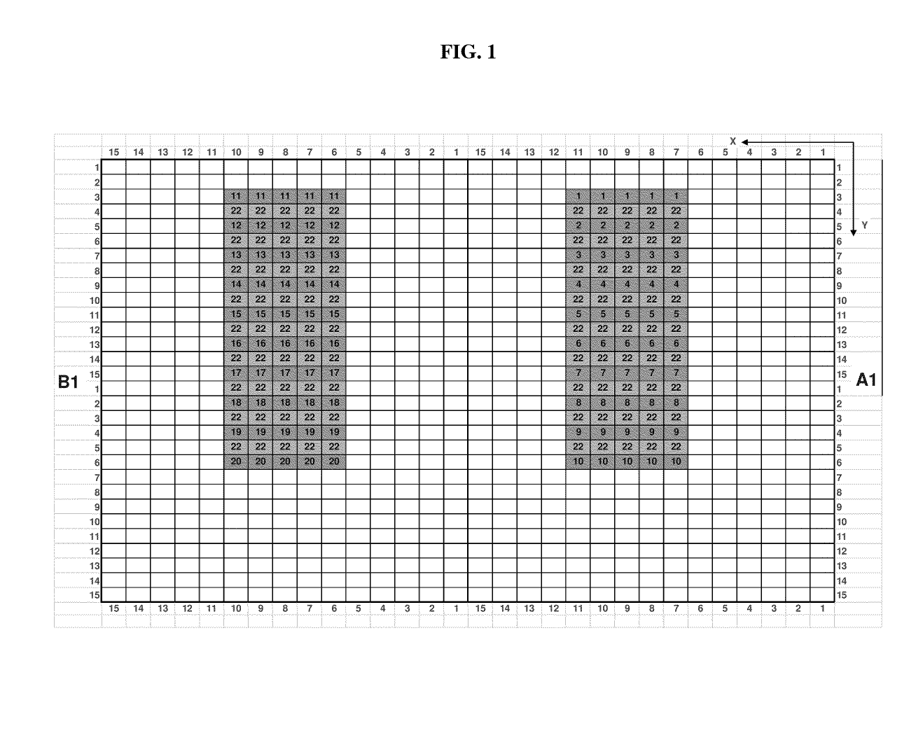

4. Increased confidence for SNP detection through physical and enzymatic action rather than by physical means alone. The following is a conceptual example of CataCleave probe technology when applied to microarray analysis. In this example two different labeled probe specific targets were spotted on a microscope slide in positions 1-20 as shown in FIG. 1. The spots labeled “22” are an amino acid mixture that was interspersed between the spots to act as a barrier to prevent cross reaction between areas of different probe concentrations.

Target Nos. 1-10 have the sequence of 5′-TGC AGC GAG CrArT rGrTT CTG GAA AGC -3′ (in which “r” indicates that the nucleotide following the letter is a ribonucleotide), which is specific for Salmonella spp., labeled with FAM at 5′-end and TAMRA at 3′-end, and each is in a concentration of 0.488, 0.977, 1.953, 3.906, 7.813, 15.63, 31.25, 62.6, 125, and 250 pmol/μl, respectively. Target Nos. 11-20 have the sequence of 5′-AAT AAA TTT GCG GrArA rCrAA AAC CAT GTG CAA-3′, (in which “r” indicates that the nucleotide following the letter is a ribonucleotide), which is specific for E. coli. O157:H7, labeled with FAM at 5′-end and TAMRA at 3′-end, and each is in a concentration of 0.488, 0.977, 1.953, 3.906, 7.813, 15.63, 31.25, 62.6, 125, and 250 pmol/μl, respectively. Primers used in the experiments have the following sequences.

Primers 1-10:
Forward 5′-TTG ATG TGG TTG GTT CGT CAC T
Reverse 5′- TCC CTG AAT CTG AGA AAG AAA AAC TC
Primers 11-20:
Forward 5′-TCA AAA GGA AAC TAT ATT CAG AAG TTT GA
Reverse 5′-CGA TAT ACC TAA CGC TAA CAA AGC TAA

The targets were labeled with a fluorescence donor at the 5′ end and a quencher at the 3′ end as explained above. Targets were spotted at different concentrations on the slide as indicated in FIG. 1. An amino acid mixture was interspersed between the spots to act as a barrier to prevent cross reaction. A solution containing single stranded probe was prepared by asymmetric PCR.

### Experimental Conditions:

Asymmetric PCR was performed under the following conditions:
5 pmol of forward primer
50 pmol reverse primer
2.5 ul 10× PCR reaction buffer
2.5 U Taq DNA polymerase
200 uM dNTP's
3 mM MgCl₂
10,000 copies of either Salmonella or E. Coli genomic DNA.
H₂O to 25 ul

Cycling Conditions
95° C. for 15 seconds
55° C. for 15 seconds
65° C. for 30 seconds
Repeat for 50 cycles
Use 5 ul of PCR product in microarray experiments.

Microarray Conditions
Microarrays were spotted using a Bio-Rad VersArray ChipWriter system according to the manufacturer's recommendations. Probe was incubated with probe specific target and 5 U of Pyrococcus furiosis RNase HII at 55 C for 60 minutes in a reaction buffer containing
32 mM HEPES-KOH, pH 7.8
100 mM KOAc
0.11% bovine serum albumin
1% dimethylsulfoxide
4 mM Mg(OAc)₂
After 60 minutes the slide was washed 5× with a reaction buffer.

The target fluorescein emission intensity was quantified using a Bio-Rad ChipReader™ system according to the manufacturer's recommendations.

Probe sequence for Targets 1-10:
5′GATGTGGTTGGTTCGTCACTGATTTTTTAGGCGCTTTTGTGCAGCGA
GCATGTTCTGGAAAGCCTCTTTATATAGCTCATTCTGACCTCTAAGC
CGGTCAATGAGTTTTTCTTTCTCAGATTCAGGGA
Probe sequence for Targets 11-20:
5′-TCAAAAGGAAACTATATTCAGAAGTTTGAAAATAAATTTGCGGAAC
AAAACCATGTGCAATATGCAACTACTGTAAGTAATGGAACGGTTGC
TCTTCATTTAGCTTTGTTAGCGTTAGGTATATCG

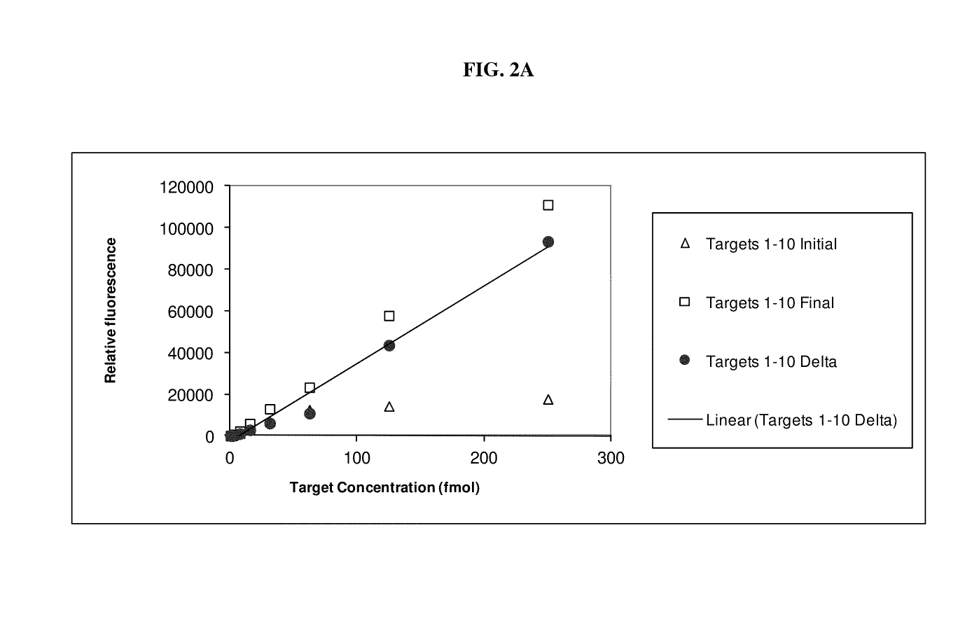

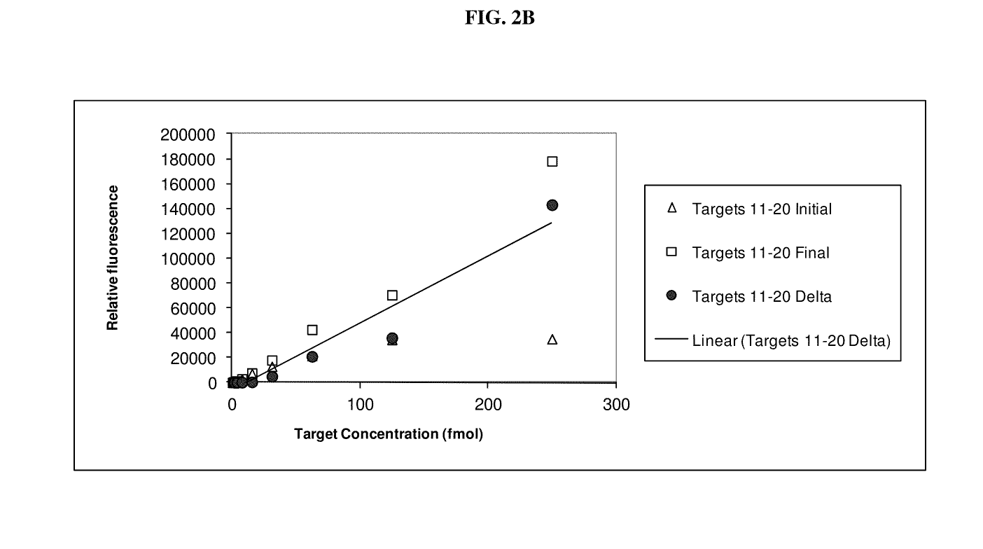

Fluorescence intensity at each index was calculated both before and after reaction using an identically spotted area to which the same reaction solution was added except that the probe is not included. The result of the experiment is presented in FIGS. 2A and 2B. In FIGS. 2A and 2B baseline fluorescence from the negative control (Initial signal) was subtracted from the probe containing sample (Final) to calculate the corrected signal (Delta).

In FIGS. 2A and 2B, it can be seen that the addition of solution containing probe and RNase HI leads to an increase in target fluorescence emission in a concentration dependent manner. This indicates that CataCleave probe can be used in a microarray application for the sensitive detection of specific target nucleic acid sequences.

1. A method of detecting a probe nucleic acid molecule in a sample, the method comprising:
a) providing an array comprising a plurality of probe specific targets wherein each probe specific target comprises: (i) a 5′-end DNA segment, (ii) a 3′-end DNA segment, and (iii) an RNA sequence within the molecule; and wherein each of the 5-end of the probe specific target array nucleic acid molecules is coupled to a detectable label, said DNA and RNA segments of the target being substantially complimentary to the probe nucleic acid, wherein the RNA sequence of the target is capable of being cleaved by the cleaving agent and a cleavage of the RNA sequence in the target results in an emission of a detectable signal from the label;
b) bringing a sample containing probe nucleic to be contacted with the probe specific target array in the presence of a mixture containing a reaction buffer and cleaving agent under conditions where the RNA sequence within the target forms a RNA:DNA heteroduplex with the complimentary sequence in the probe nucleic acid; and
c) detecting a change in the emission of a signal from the label of the target.

2. The method according to claim 1, wherein the change in the emission of the signal is an increase in signal.

3. The method according to claim 1, wherein the RNA segment in the probe specific target is composed of 1-6 bases.

4. The method according to claim 1, wherein the detectable label is a FRET pair.

5. A method for real-time detection of a polymorphism in a probe DNA, comprising:
a) providing a probe sample to be tested for the presence of a target specific DNA, providing a probe specific target comprising a detectable label and DNA and RNA nucleic acid sequences, wherein the probe specific targets RNA nucleic acid sequences are entirely complementary to a selected region of the probe DNA and the probe specific targets DNA nucleic acid sequences are substantially complementary to DNA sequences adjacent to the selected region of the probe DNA sequence, wherein the probe specific target is immobilized on a surface of a solid support; in the presence of an reaction buffer; an RNase H activity and the probe under conditions where the RNA sequences within the probe specific target can form a RNA:DNA heteroduplex with the complementary DNA sequences in the probe PCR fragment, and
b) detecting a real-time increase in the emission of a signal from the label on the probe specific target, wherein the increase in signal indicates the presence of the probe DNA.

6. The method according to claim 5, wherein the real-time increase in the emission of the signal from the label on the probe specific target results from the RNase H cleavage of the targets RNA sequences in the RNA:DNA heteroduplex.

7. The method according to claim 5, wherein the detectable label is a FRET pair.

8. A method for real-time detection of a polymorphism in a probe DNA, comprising:
a) providing a probe sample to be tested for the presence of a target specific DNA having a polymorphism,
b) providing a probe specific target comprising a detectable label and DNA and RNA nucleic acid sequences, wherein the probe specific targets RNA nucleic acid sequences are entirely complementary to a selected region of the probe DNA sequence comprising a wild type DNA sequence at the location of the polymorphism and the probe specific targets DNA nucleic acid sequences are substantially complementary to DNA sequences adjacent to the selected region of the probe DNA sequence, wherein the probe specific target is immobilized on a surface of a solid support, in the presence of a reaction buffer; an RNase H activity and the probe specific target under conditions where the RNA sequences within the probe specific target can form a RNA:DNA heteroduplex with the complementary DNA sequences in the probe PCR fragment comprising the polymorphism, and
c) detecting a real-time decrease in the emission of a signal from the label on the probe specific target, wherein the decrease in signal indicates the presence of the polymorphism in the probe DNA.

9. The method according to claim 8, wherein the real-time increase in the emission of the signal from the label on the probe specific target results from the RNase H cleavage of the probe specific targets RNA sequences in the RNA:DNA heteroduplex.

10. The method according to claim 8, wherein the detectable label is a FRET pair.

11. A method of detecting a probe nucleic acid molecule in a sample, the method comprising:
a) providing an array comprising a plurality of probe specific targets wherein each probe specific target comprises: (i) a 5′-end DNA segment, (ii) a 3′-end DNA segment, and (iii) an RNA sequence within the molecule; and wherein each of the 5-end of the probe specific target specific array nucleic acid molecules is coupled to a detectable label, said DNA and RNA segments of the probe specific target being substantially complimentary to the probe nucleic acid, wherein the RNA sequence of the probe specific target is capable of being cleaved by the cleaving agent and a cleavage of the RNA sequence in the probe specific target results in an emission of a detectable signal from the label;
b) bringing a probe sample to be contact with the probe specific target array in the presence of a mixture containing a first primer oligonucleotide, a second primer oligonucleotide, a polymerase activity, a cleaving agent, and deoxynucleoside triphosphates wherein the first primer oligonucleotide and the second oligonucleotide can anneal to the probe nucleic acid, under conditions where the RNA sequence within the probe specific target forms a RNA:DNA heteroduplex with the complimentary sequence in the probe nucleic acid; and
c) detecting a change in the emission of a signal from the label of the probe specific target.

12. The method according to claim 11, wherein the change in the emission of the signal is an increase in signal.

13. The method according to claim 11, wherein the RNA segment in the probe specific target is composed of 1-6 bases.

14. The method according to claim 11, wherein the detectable label is a FRET pair.

15. A kit for real-time detection of a polymorphisms in a probe DNA comprising:
a) a pair of amplification primers that can anneal to a probe DNA, wherein a first amplification primer anneals upstream of the location of a polymorphism and a second amplification primer anneals downstream of the location of the polymorphism;
b) a plurality of probe specific targets each comprising a detectable label and DNA and RNA nucleic acid sequences, wherein the probe specific targets RNA nucleic acid sequences are entirely complementary to a selected region of the probe DNA sequence comprising the polymorphism and the probe specific targets DNA nucleic acid sequences are substantially complementary to DNA sequences adjacent to the selected region of the probe DNA sequence, wherein the probe specific target is immobilized on a surface of a solid support; and
c) an RNase H activity.

16. The kit according to claim 15, wherein the detectable label is a FRET pair.

17. The kit according to claim 15, which further comprises
d) an amplifying polymerase activity; and
e) amplification buffer.

18. A kit for real-time detection of a polymorphism in a probe DNA comprising
a) a pair of amplification primers that can anneal to a probe DNA, wherein a first amplification primer anneals upstream of the location of a polymorphism and a second amplification primer anneals downstream of the location of the polymorphism; and
b) a plurality of probe specific targets each comprising a detectable label and DNA and RNA nucleic acid sequences, wherein the probe specific targets RNA nucleic acid sequences are entirely complementary to a selected region of the probe DNA sequence comprising the wild type DNA sequence at the location of the polymorphism and the probe specific targets DNA nucleic acid sequences are substantially complementary to DNA sequences adjacent to the selected region of the probe DNA sequence, wherein the probe specific targets are immobilized on a surface of a solid support,
an amplifying polymerase activity.

19. The kit according to claim 18, wherein the detectable label is a FRET pair.

20. The kit according to claim 19, which further comprises
d) an amplifying polymerase activity; and
e) amplification buffer.


SEQUENCE LISTING (8 sequences)

SEQ ID NO: 1 - DNA, 24 bp, Artificial Sequence
  Chimeric polynucleotide with 2′-deoxynucleotide and 2′-oxynucleotide.
tgcagcgagcatgttctggaaagc

SEQ ID NO: 2 - DNA, 30 bp, Artificial Sequence
  Chimeric polynucleotide with 2′-deoxynucleotide and 2′-oxynucleotide.
aataaatttgcggaacaaaaccatgtgcaa

SEQ ID NO: 3 - DNA, 22 bp, Artificial Sequence
  Forward primer
ttgatgtggttggttcgtcact

SEQ ID NO: 4 - DNA, 26 bp, Artificial Sequence
  Reverse primer
tccctgaatctgagaaagaaaaactc

SEQ ID NO: 5 - DNA, 29 bp, Artificial Sequence
  Forward primer
tcaaaaggaaactatattcagaagtttga

SEQ ID NO: 6 - DNA, 27 bp, Artificial Sequence
  Reverse primer
cgatatacctaacgctaacaaagctaa

SEQ ID NO: 7 - DNA, 128 bp, Artificial Sequence
  Probe sequence for targets 1-10
gatgtggttggttcgtcactgattttttaggcgcttttgtgcagcgagcatgttctggaaagcctctttatatagctcattctgacctctaagccggtcaatgagtttttctttctcagattcaggga

SEQ ID NO: 8 - DNA, 126 bp, Artificial Sequence
  Probe sequence for targets 11-20
tcaaaaggaaactatattcagaagtttgaaaataaatttgcggaacaaaaccatgtgcaatatgcaactactgtaagtaatggaacggttgctcttcatttagctttgttagcgttaggtatatcg